(19) United States
(12) Patent Application Publication
(10) Pub. No.: US 2024/0415577 A1
(43) Pub. Date: Dec. 19, 2024

(54) System And Method For Navigation

(71) Applicant: Medtronic Navigation, Inc., Lafayette, CO (US)

(72) Inventor: Bradley W. Jacobsen, Erie, CO (US)

(21) Appl. No.: 18/675,355

(22) Filed: May 28, 2024

Related U.S. Application Data
(60) Provisional application No. 63/508,628, filed on Jun. 16, 2023.

(51) Int. Cl. A61B 34/20

(52) U.S. Cl. CPC A61B 34/20; A61B 2034/2051; A61B 2034/2063

(57) ABSTRACT

Disclosed is a system for assisting in guiding and performing a procedure on a subject. The subject may be any appropriate subject such as inanimate object and/or an animate object.

### CROSS-REFERENCE TO RELATED APPLICATION

This application claims the benefit of and priority to U.S. Provisional Patent Application No. 63/508,628 filed Jun. 16, 2023, the entire disclosure of which is incorporated by reference herein.

### FIELD

The subject disclosure is related generally to a tracking and navigation system, and particularly to tracking using an electromagnetic field and sensor.

### BACKGROUND

This section provides background information related to the present disclosure which is not necessarily prior art.

An instrument can be navigated relative to a subject for performing various procedures. For example, the subject can include a patient on which a surgical procedure is being performed. During a surgical procedure, an instrument can be tracked in a physical space which may also be referred to as an object or subject space. In various embodiments, the subject space can be a patient space defined by a patient. The location of the instrument that is tracked can be displayed on a display device relative to an image of the patient.

The position of the patient can be determined with a tracking system. Generally, a patient is registered to the image, via tracking an instrument relative to the patient to generate a translation map between the subject or object space (e.g., patient space) and the image space. This often requires time during a surgical procedure for a user, such as a surgeon, to identify one or more points in the subject space and correlating, often identical points, in the image space.

After registration, the position of the instrument can be appropriately displayed on the display device while tracking the instrument. The position of the instrument relative to the subject can be displayed as a graphical representation, sometimes referred to as an icon on the display device.

### SUMMARY

This section provides a general summary of the disclosure, and is not a comprehensive disclosure of its full scope or all of its features.

According to various embodiments, an imaging system may be used to acquire image data of a subject. The imaging system may include an ultrasound imaging system that includes an ultrasound (US) probe that generally includes an ultrasound transducer to emit and receive ultrasound frequencies. It is understood, however, that the imaging system may include separate components that emit and receive ultrasound frequencies.

According to various embodiments, the US probe may be moved relative to a subject, such as a by a user and/or with a robotic system. The US probe may be moved relative to the subject in any appropriate manner, however. Further, the US probe may be held relative to the subject with an appropriate holder or mount. Regardless, various objects may be placed relative to the subject, such as in or near the subject space. The various objects may be formed of various materials such as conductive materials including metal or metal alloys.

An object, also referred to as an interfering object, formed of conductive materials may have currents induced therein due to fields that are formed near the object having the conductive materials. The conductive materials may also be referred to as interfering materials. A field, such as from a tracking system electromagnetic field, may induce currents within the conductive materials, such as Eddy currents. The conductive materials or objects may thereafter emit fields that are not emitted by the tracking system. The fields that are emitted by the conductive objects or materials may interfere with the field emitted by the electromagnetic tracking system. Therefore, the various objects formed of conductive materials may form interfering fields. The interfering fields may interfere with tracking of a tracking device.

Prior to tracking a tracking device, an object may have various designs formed therein. An interfering object may be augmented such that the interfering object emits reduced, reduced and canceling, minimized, or no interfering fields. The interfering object may have its surface cut or otherwise augmented to reduce or minimize interfering fields that may be produced if the interfering object is not augmented. Augmentations may include cuts or grooves formed in the interfering objects, such as into a surface and/or in a portion thereof.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is diagrammatic view illustrating an overview of a robotic system and a navigation system, according to various embodiments;

FIG. 2 is an exemplary interfering object having an augmentation, according to various embodiments;

FIG. 3 is a cross-sectional view taken along line 3-3 of FIG. 2;

FIG. 4 is a cross-sectional view taking a long line 4-4 of FIG. 2;

FIG. 4′ is a cross-sectional view taken the long line 4′-4′ of FIG. 2;

FIG. 5 is an exemplary view of an interfering object having an augmentation, according to various embodiments;

FIG. 6 is a cross-sectional view taken along line 6-6 of FIG. 5; and

FIG. 7 is an exemplary view of an ultrasound probe, according to various embodiments.

Corresponding reference numerals indicate corresponding parts throughout the several views of the drawings.

### DETAILED DESCRIPTION

Example embodiments will now be described more fully with reference to the accompanying drawings.

The subject disclosure is directed to an exemplary embodiment of a surgical procedure on a subject, such as a human patient. It is understood, however, that the system and methods described herein are merely exemplary and not intended to limit the scope of the claims included herein. In various embodiments, it is understood, that the systems and methods may be incorporated into and/or used on non-animate objects. The systems may be used to, for example, to register coordinate systems between two systems for use on manufacturing systems, maintenance systems, and the like. For example, automotive assembly may use one or more robotic systems including individual coordinate systems that may be registered together for coordinated or concerted actions. Accordingly, the exemplary illustration of a surgical procedure herein is not intended to limit the scope of the appended claims.

Discussed herein, according to various embodiments, is a tracking system that may be used to track a selected tracking device. The tracking system may operate, according to various embodiments, by emitting an electromagnetic (EM) field from a localizer, also referred to as an EM localizer. The EM field may be emitted from one or more coils that may be oriented relative to an origin point. The coils may emit the field. The field may be a largely magnetic field. The field may be constant or varying in time. A tracking device may include one or more coils that operate as sensors to sense the field. The field may generate a current within the coil of the tracking device. A determination of a position and orientation (also referred to collectively as a “pose”) of the tracking device may be made.

Various materials are conductive, such as conductive polymers, metal or metal alloys, or other materials. Objects or items may be formed with these materials. If an item formed with these materials is also in or near the field generated by the EM localizer, a current may be formed or induced in the object. In this instance, the object may be referred to as an interfering object. When a current is induced in the interfering object, a field may also be produced. A field produced due to the induced current in the interfering object may also be referred to as an interfering field. These interfering fields may alter the field sensed by the tracking device such that it is not always sensing the EM field generated by the EM localizer.

According to various embodiments, however, as discussed further herein in various examples, the interfering objects may be augmented. An augmentation of the interfering object may include forming a cut or groove in a portion of the interfering object. For example, the interfering object may include an exterior case or a portion that defines a surface. A groove may be formed in the surface, or the surface may be cut such that it is formed into a plurality of pieces. The fields induced in an augmented interfering object, however, may be reduced in general, may partially cancel in general, and may fully cancel at certain geometries and so may minimally or not interfere with the field produced by the EM localizer. Therefore, the augmented object may produce minimally interfering fields or no fields, according to various embodiments.

Various portions may be tracked relative to the subject. For example, a tracking system may be incorporated into a navigation system that includes one or more instruments that may be tracked relative to the subject. The navigation system may include one or more tracking systems that track various portions, such as tracking devices, associated with instruments. The tracking system may include a localizer that is configured to, alone or in combination with a processor, determine the pose of a tracking device in a navigation system coordinate system. Determination of the navigation system coordinate system may include those described at various references including U.S. Pat. Nos. 8,737,708; 9,737,235; 8,503,745; and 8,175,681; all incorporated herein by reference. In particular, a localizer may be able to track an object within a volume relative to the subject. The navigation volume, in which a device may be tracked may include or be referred to as the navigation coordinate system or navigation space. A determination or correlation between two coordinate systems may allow for or also be referred to as a registration between two coordinate systems.

Furthermore, images may be acquired of selected portions of a subject. The images may be displayed for viewing by a user, such as a surgeon. The images may have superimposed on a portion of the image can include a graphical representation of a tracked portion or member, such as an instrument. The images may have a coordinate system and define an image space. According to various embodiments, the graphical representation may be superimposed on the image at an appropriate position due to registration of an image space (also referred to as an image coordinate system) to a subject space. A method to register a subject space defined by a subject to an image space may include those disclosed in U.S. Pat. Nos. 8,737,708; 9,737,235; 8,503,745; and 8,175,681; all incorporated herein by reference.

During a selected procedure, a coordinate system may be registered to the subject space or subject coordinate system due to a selected procedure, such as imaging of the subject. In various embodiments, the first coordinate system may be registered to the subject by imaging the subject with a fiducial portion that is fixed relative to the first member or system, such as the robotic system. The known position of the fiducial relative to the robotic system may be used to register the subject space relative to the robotic system due to the image of the subject including the fiducial portion. Thus, the position of the robotic system or a portion thereof, such as the end effector, may be known or determined relative to the subject. Due to registration of a second coordinate system to the robotic coordinate system may allow for tracking of additional elements not fixed to the robot relative to a position determined or tracked by the robot.

The tracking of an instrument during a procedure, such as a surgical or operative procedure, allows for navigation of a procedure. When image data is used to define an image space it can be correlated or registered to a physical space defined by a subject, such as a patient as discussed herein. According to various embodiments, therefore, the patient defines a patient space in which an instrument can be tracked and navigated. The image space defined by the image data can be registered to the patient space defined by the patient. The registration can occur with the use of fiducials that can be identified in the image data and in the patient space.

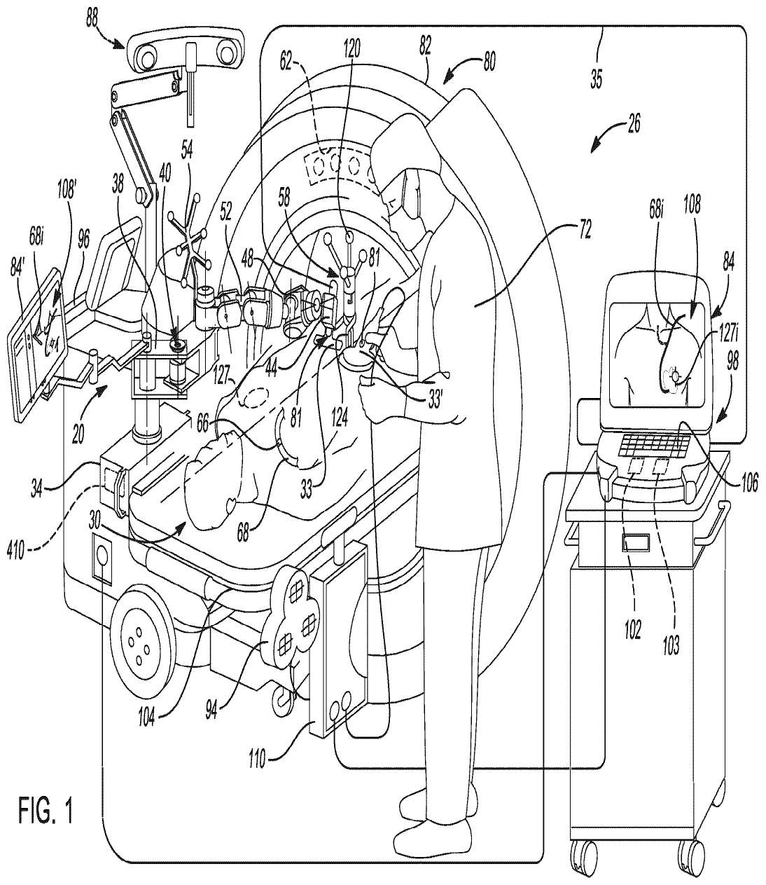

FIG. 1 is a diagrammatic view illustrating an overview of a procedure room or arena. In various embodiments, the procedure room may include a surgical suite in which may be placed a robotic system 20 and a navigation system 26 that can be used for various procedures. The robotic system 20 may include a Mazor X™ robotic guidance system, sold by Medtronic, Inc. The robotic system 20 may be used to assist in guiding a selected instrument, such as drills, screws, etc. relative to a subject 30. In addition or alternatively, the robotic system 20 may hold and/or move an imaging system, such as an ultrasound (US) probe 33. The robotic system 20 may include a mount 34 that fixes a portion, such as a robotic base 38, relative to the subject 30. The robotic system 20 may include one or more arms 40 that are moveable or pivotable relative to the subject 30, such as including an end effector 44. The end effector may be any appropriate portion, such as a tube, guide, or passage member. Affixed to and/or in place of the end effector may be the imaging system that may be the US probe 33. The end effector 44 may be moved relative to the base 38 with one or more motors. The position of the end effector 44 may be known or determined relative to the base 38 with one or more encoders at one or more joints, such as a wrist joint 48 and/or an elbow joint 52 of the robotic system 20. One or more portions of the robotic system 20 may be formed of conductive materials.

The navigation system 26 can be used to track the location of one or more tracking devices and/or determine and/or illustrate a pose thereof. Tracking devices may include a robot tracking device 54, a subject tracking device 58, an imaging system tracking device 62, an imaging system or second imaging system tracking device 81, and/or an instrument or tool tracking device 66. A tool or moveable member 68 may be any appropriate tool such as a drill, forceps, catheter, or other tool operated by a user 72. The tool 68 may also include an implant, such as a spinal implant or orthopedic implant. It should further be noted that the navigation system 26 may be used to navigate any type of instrument, implant, or delivery system, including: guide wires, arthroscopic systems, orthopedic implants, spinal implants, deep brain stimulation (DBS) probes, etc. Moreover, the instruments may be used to navigate or map any region of the body. The navigation system 26 and the various instruments may be used in any appropriate procedure, such as one that is generally minimally invasive or an open procedure.

An additional or alternative, imaging system 80 may be used to acquire pre-, intra-, or post-operative or real-time image data of a subject, such as the subject 30. It will be understood, however, that any appropriate subject can be imaged and any appropriate procedure may be performed relative to the subject. In the example shown, the imaging system 80 comprises an O-Arm® imaging device sold by Medtronic Navigation, Inc. having a place of business in Colorado, USA. The imaging system 80 may have a generally annular gantry housing 82 in which an image capturing portion is moveably placed and/or enclosed. The imaging system 80 can include those disclosed in U.S. Pat. Nos. 7,188,998; 7,108,421; 7,106,825; 7,001,045; and 6,940,941; all of which are incorporated herein by reference, or any appropriate portions thereof. It is further appreciated that the imaging system 80 may include in addition or alternatively a fluoroscopic C-arm. Other exemplary imaging devices may include fluoroscopes such as bi-plane fluoroscopic systems, ceiling mounted fluoroscopic systems, cath-lab fluoroscopic systems, fixed C-arm fluoroscopic systems, isocentric C-arm fluoroscopic systems, 3D fluoroscopic systems, etc. Other appropriate imaging devices can also include MRI, CT, ultrasound, etc.

The position of the imaging system 33, 80, and/or portions therein such as the image capturing portion, can be precisely known relative to any other portion of the imaging device 33, 80. The imaging device 33, 80, according to various embodiments, can know and/or recall precise coordinates relative to a fixed or selected coordinate system. For example, the robotic system 20 may know or determine its position and position the US probe 33 at a selected pose. Similarly, the imaging system 80 may also position the imaging portions at a selected pose. This can allow the imaging system 80 to know its position relative to the patient 30 or other references. In addition, as discussed herein, the precise knowledge of the position of the image capturing portion can be used in conjunction with a tracking system to determine the position of the image capturing portion and the image data relative to the tracked subject, such as the patient 30. In other words, the imaging system tracking device 62, 81 may be used and/or operable to determine a pose of the imaging system 33, 80 at a selected time such as during image data acquisition.

Herein, reference to the imaging system 33 may refer to any appropriate imaging system, unless stated otherwise. Thus, the US probe 33 as the imaging system is merely exemplary regarding the subject disclosure. As one skilled in the art will understand, generally the US probe 33 may emit a US wave in a plane and receive an echo relative to any portions engaged by the wave. The received echo at the US probe 33 or other appropriate received may be used to generate image data and may be used to generate an US image also referred to as a sonogram.

The imaging device 80 can be tracked with the tracking device 62. Also, the tracking device 81 can be associated directly with the US probe 33. The US probe 33 may, therefore, be directly tracked with a navigation system 26 as discussed herein. In addition or alternatively, the US probe 33 may be positioned and tracked with the robotic system 20. Regardless, image data defining an image space acquired of the patient 30 can, according to various embodiments, be registered (e.g., manually, inherently, or automatically) relative to an object space. The object space can be the space defined by a patient 30 in the navigation system 26.

The patient 30 can also be tracked as the patient moves with a patient tracking device, DRF, or tracker 58. Alternatively, or in addition thereto, the patient 30 may be fixed within navigation space defined by the navigation system 26 to allow for and/or maintain registration such as to the image space of the image 108. As discussed further herein, registration of the image space to the patient space or subject space allows for navigation of the instrument 68 with the image data. When navigating the instrument 68, a position of the instrument 68 can be illustrated relative to image data acquired of the patient 30 on a display device 84 such as with a graphical representation 68i, 68i′. An additional and/or alternative display device 84′ may also be present to display an image. Various tracking systems, such as one including an optical localizer 88 or an electromagnetic (EM) localizer 92 can be used to track the instrument 68.

More than one tracking system can be used to track the instrument 68 or other portion, such as the US probe 33 with the tracking device 81 in the navigation system 26. According to various embodiments, these can include an electromagnetic tracking (EM) system having the EM localizer 94 and/or an optical tracking system having the optical localizer 88. Either or both of the tracking systems can be used to track selected tracking devices, as discussed herein. It will be understood, unless discussed otherwise, that a tracking device can be a portion trackable with a selected tracking system. A tracking device need not refer to the entire member or structure to which the tracking device is affixed or associated.

The position of the patient 30 relative to the imaging device 33 can be determined by the navigation system 26. The position of the imaging system 33 may be determined, as discussed herein. The patient 30 can be tracked with the dynamic reference frame 58, as discussed further herein. Accordingly, the position of the patient 30 relative to the imaging device 33 can be determined.

Image data acquired from the imaging system 33, or any appropriate imaging system, can be acquired at and/or forwarded from an image device controller 96, that may include a processor module, to a navigation computer and/or processor module (also referred to as a processor) 102 that can be a part of a controller or work station 98 having the display 84 and a user interface 106. Further, a memory 103, of any appropriate type, may be accessed by the processor 102. It will also be understood that the image data is not necessarily first retained in the controller 96, but may also be directly transmitted to the work station 98. The work station 98 can provide facilities for displaying the image data as an image 108 on the display 84, saving, digitally manipulating, or printing a hard copy image of the received image data. The user interface 106, which may be a keyboard, mouse, touch pen, touch screen or other suitable device, allows the user 72 to provide inputs to control the imaging device 80, 33, via the image device controller 96, or adjust the display settings of the display 84. The work station 98 may also direct the image device controller 96 to adjust the image capturing portion of the imaging device 80 to obtain various two-dimensional images along different planes in order to generate representative two-dimensional and three-dimensional image data.

With continuing reference to FIG. 1, the navigation system 26 can further include the tracking system including either or both of the electromagnetic (EM) localizer 94 and/or the optical localizer 88. The tracking systems may include a controller and interface portion 110. The controller 110 can be connected to the processor portion 102, which can include a processor included within a computer. The EM tracking system may include the STEALTHSTATION® AXIEM™ Navigation System, sold by Medtronic Navigation, Inc. having a place of business in Louisville, Colorado; or can be the EM tracking system described in U.S. patent application Ser. No. 10/941,782, filed Sep. 15, 2004, and entitled “METHOD AND APPARATUS FOR SURGICAL NAVIGATION”; U.S. Pat. No. 5,913,820, entitled “Position Location System,” issued Jun. 22, 1999; and U.S. Pat. No. 5,592,939, entitled “Method and System for Navigating a Catheter Probe,” issued Jan. 14, 1997; all of which are herein incorporated by reference. It will be understood that the navigation system 26 may also be or include any appropriate tracking system, including a STEALTHSTATION® TREON® or S7™ tracking systems having an optical localizer, that may be used as the optical localizer 88, and sold by Medtronic Navigation, Inc. of Colorado. Other tracking systems include an acoustic, radiation, radar, etc. The tracking systems can be used according to generally known or described techniques in the above incorporated references. Details will not be included herein except when to clarify selected operation of the subject disclosure.

Wired or physical connections can interconnect the tracking systems, imaging device 80, etc. Alternatively, various portions, such as the instrument 68 may employ a wireless communications channel, such as that disclosed in U.S. Pat. No. 6,474,341, entitled “Surgical Communication Power System,” issued Nov. 5, 2002, herein incorporated by reference, as opposed to being coupled directly to the controller 110. Also, the tracking devices 62, 66, 54 can generate a field and/or signal that is sensed by the localizer(s) 88, 94.

Various portions of the navigation system 26, such as the instrument 68, and others as will be described in detail below, can be equipped with at least one, and generally multiple, of the tracking devices 66. The instrument can also include more than one type or modality of tracking device 66, such as an EM tracking device and/or an optical tracking device. The instrument 68 can include a graspable or manipulable portion at a proximal end and the tracking devices may be fixed near the manipulable portion of the instrument 68.

Additional representative or alternative localization and tracking system is set forth in U.S. Pat. No. 5,983,126, entitled “Catheter Location System and Method,” issued Nov. 9, 1999, which is hereby incorporated by reference. The navigation system 26 may be a hybrid system that includes components from various tracking systems.

According to various embodiments, the navigation system 26 can be used to track any appropriate portion such as the US probe 33 and/or the instrument 68 relative to the patient 30. The instrument 68 can be tracked with the tracking system, as discussed above. Image data of the patient 30, or an appropriate subject, can be used to assist the user 72 in guiding the instrument 68. The image data may or may not be registered to the patient 30. For example, as discussed herein, the US probe 33 is tracked and generates the image data. Thus, the image data need not be registered to the subject to display a pose of the tracked instrument 68 relative to the image data generator with the tracked US probe 33. The image data defines the image space that is registered to the patient space defined by the patient 30. The registration can be performed as discussed herein, automatically, manually, or combinations thereof. The registration can include the process and the final transformation (including a translation and rotation) map. Generally, registration includes determining points in the image data and the subject space and determining a transformation map therebetween. Once done, the image space are registered to the subject space, or any two or more coordinate spaces.

Generally, registration also allows a transformation map to be generated of a tracked physical pose of the instrument 68 relative to the image space of the image data. The transformation map allows the tracked position of the instrument 68 to be displayed on the display device 84 relative to the image data 108. The graphical representation 68i, also referred to as an icon, can be used to illustrate the location of the instrument 68 relative to the image data 108.

With continuing reference to FIG. 1, a subject registration system or method can use the tracking device 58. The tracking device 58 may include portions or members 120 that may be trackable, but may also act as or be operable as a fiducial assembly. The fiducial assembly 120 can include a clamp or other fixation portion 124 and the imagable fiducial body 120. It is understood, however, that the members 120 may be separate from the tracking device 58. The fixation portion 124 can be provided to fix any appropriate portion, such as a portion of the anatomy. As illustrated in FIG. 1, the fiducial assembly 120 can be interconnected with a portion of a spine 126 such as a spinous process 130. The fixation portion 124 can be interconnected with a spinous process 130 in any appropriate manner. For example, a pin or a screw can be driven into the spinous process 130. Further, the tracking device 58 may be operable to track with one or more tracking systems or modalities, such as EM tracking system or optical tracking system.

As illustrated in FIG. 1, the imaging device 33 may include the US probe 33 that may be positioned relative to the subject 30, such as by the robotic system 20 and/or the surgeon 72. In various embodiments, the surgeon 72 may operate the robotic arm 20 and/or hold the US probe 33 separate therefrom. As discussed herein, therefore, the robotic system 20 may move the US probe 33 to a selected position relative to the subject 30. According to various embodiments, the imaging system may be positioned relative to the subject in any appropriate manner.

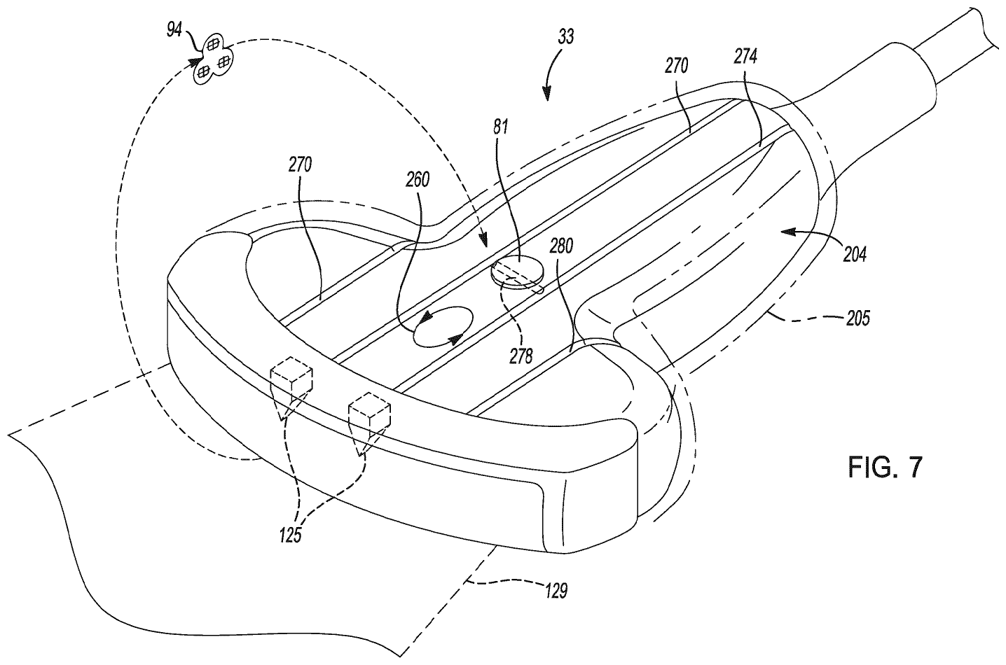

Further, as is understood by one skilled in the art, the image data acquired with one or more ultrasound arrays 125 (FIG. 7) of the US probe 33 may be registered in navigation system such as disclosed in the U.S. Pat. Nos. 7,085,400 and 9,138,204, both incorporated herein by reference. The image data acquired within the respective ultrasound arrays 125 may be of the subject 30. As the ultrasound arrays 125 are registered to the subject 30 using the navigation system 26, the image data required of the subject 30, such as of a heart 127 and/or other subject portion such as a vertebrae, may also have its pose determined in navigation space within the navigation system 26. The image data may be used to generate a specific image portion such as an image of the heart 127i. The image 127i may be a reconstruction based on the image data from the US probe 33. The graphical representation 68i may be represented relative to the image 108 and/or portions therefore such as the image of the heart 127i. Further, the graphical representation 68i may be superimposed on the reconstruction and/or the image. The reconstruction may include additional data (e.g., atlas or population data) and may also be referred to as a model. The graphical representation may be superimposed on the model.

Briefly, conductive objects, e.g., metallic objects including conductive metals, may be specifically patterned or formed to maintain the structure and function of the object and to reduce EM distortions, also referred to as interfering fields. Specific patterns for conductive metals may include but are not limited to cuts of surfaces (areas) or bodies (volumes) or combinations thereof to reduce areas or volumes of induced currents, increase effective cancelation of induced currents, reduce strengths of induced distorting (also referred to as interfering) magnetic fields, and reduce reaches of induced distorting magnetic fields. Specifically patterned conductive metallic objects may enable accurate EM navigation of those objects using exterior, near, attached, upon, interior, inside, embedded, or surrounded EM tracking devices.

The objects may have patterns applied to but are not limited to parts of or entire medical devices (e.g., pacemakers or stimulators), instruments (e.g., retractors or trocars), therapy delivery systems (e.g., capsules), imaging systems (e.g., C-arm image intensifiers and bodies, O-arm gantries and bodies, or ultrasound bodies and internal structures). Various embodiments having patterns formed thereon or with are disclosed herein.

Patterns may include various shapes, according to various embodiments, including points, lines, curves, areas, volumes, and combinations thereof. Patterns may include but are not limited to cuts of surfaces (areas) or bodies (volumes) or combinations thereof. Patterns may be but are not limited to cuts of various depths including partial and full depths.

Patterns may include but are not limited to cuts which may be supported by uncut metal or other material a selected distance away from EM tracking devices. Patterns may include but are not limited to cuts which may leave a metal layer thin enough to cause high resistance for induced currents formed from navigation EM fields at lower frequencies (e.g. from 0 to approximately 30 kHz) to reduce EM distortions but thick enough to cause low resistance for induced currents from EM fields at higher frequencies (e.g. greater than approximately 30 kHz) to maintain effective EM immunity and compatibility shielding.

Patterns may control area or volume induced currents to increase effective cancelation of induced magnetic fields.

As discussed above, a field may be emitted by a selected item, such as the EM localizer 94. The EM localizer 94 may generate one or more fields with one or more coils that is (are) emitted into the volume near the localizer 94, such as including the subject 30 and other items, such as the US Probe 33. The one or more fields may be constant or time varying and/or frequency varying. In various embodiments, for example as illustrated in FIG. 7, the US Probe 33 may have supporting or other functioning structures of a conductive material 204 that may be housed or contacted within a housing 205. It is understood, however, that the housing of the US probe 33 may be formed entirely or partially of the conductive material 204. Regardless, in various embodiments, the conductive material may interact with the emitted field from the EM localizer 94 and have induced therein currents. The induced currents may generate fields that may be interfering fields, as noted above.

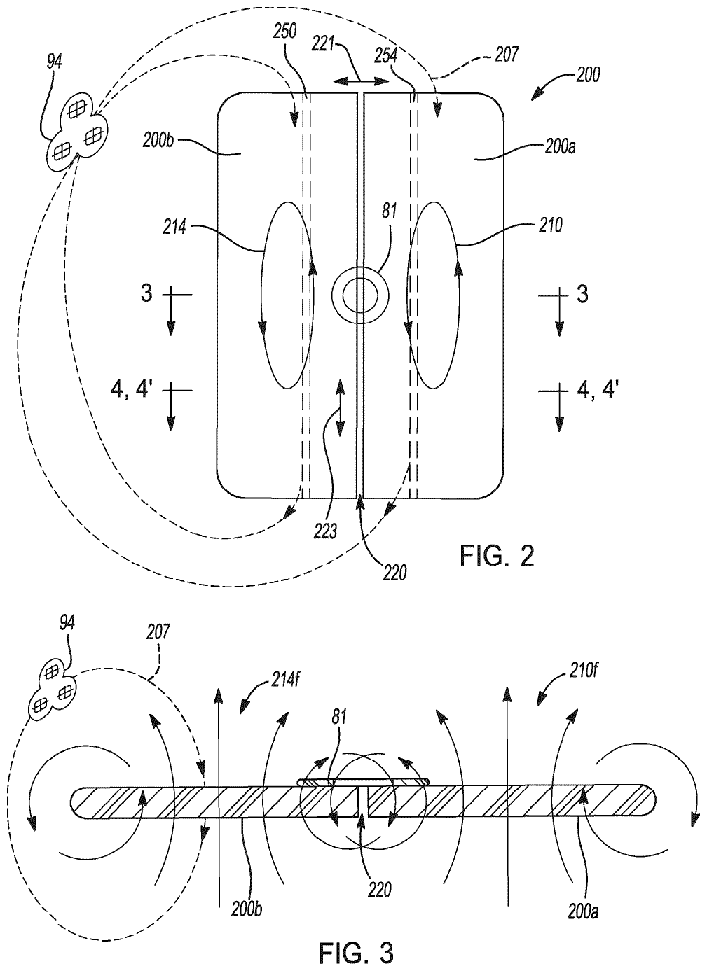

With continuing reference to FIG. 1 and additional reference to FIG. 2 and FIG. 3, a substantially flat or planar object or item 200 is illustrated. The planar item 200 may be any appropriate item, such as at least a portion (e.g., a side or surface) of a structure or portion 204 of the US Probe 33, as illustrated in FIG. 7. The planar item 200 may be formed into a single piece and/or formed of several pieces such as to form the probe portion 204. The planar item 200 illustrated in FIG. 2 is merely exemplarily of a planar item. Nevertheless, the planar item may have induced therein one or more eddy currents, such as a first eddy current represented by arrow 210 and a second eddy current represented by arrow 214. The two eddy currents (also referred to herein by the arrows 210, 214) may be formed separately due to an augmentation 220 of the planar item 200. The augmentation 220 may be formed as a pattern or gap, opening, cut, etc., or also referred to as a pattern, as discussed above, in the planar item 200.

The eddy current formed within the planar member 200, either in the individual portions 200a, 200b or in any portion thereof is generally due to the generation of an induced field from the EM localizer 94. As illustrated in FIG. 2 and FIG. 3, the EM localizer 94 may be energized to generate a field. A field, such as a time varying magnetic field may be emitted by the EM localizer 94. The field induces an electric field and induces currents in conductive objects. The induced currents may produce interfering magnetic fields, as discussed herein. The emitted EM field may be exemplified and/or represented by field line(s) 207 extending from the EM Localizer 94 and interact with the planar member 200, or portions thereof. Herein, the filed lines may be referred to as the EM field as well, for ease of the current discussion. The interaction of the field 207 with the planar item 200 may induce a current, or more than one current, in a portion of the planar item 200 due to the planar item 200 being formed of a conductive material. Therefore, the eddy currents formed therein, such as represented by the circles 210, 214 may be induced due to the EM field generated and emitted by the EM localizer 94.

The tracking device 81 may be associated with the planar item 200. For example, the tracking device 81 may be positioned on, near, inside a housing or structure formed therewith, and/or embedded in the item 200. With reference to FIG. 3, the planar item 200 may, therefore, include a first portion 200a having the eddy current 210 and the second portion 200b having the eddy current 214. The field emitted by the EM localizer 94 may induce the currents represented by the arrows 210, 214 in the respective portions 200a, 200b separated by the augmentation 220 of the planar item 200. The eddy currents in the respect of members 200a, 200b may then generate fields such as represented by the field arrows 210f and 214f relating to their respective eddy current represented by the arrows 210, 214 illustrated in FIG. 3.

The fields 210f, 214f may be interfering field if they are large enough, strong enough, and or near enough the tracking device 81. The fields 210f, 214f may not be interfering fields, however, if they are small enough and/or effectively cancel each other such as due to the augmentation 220. If the augmentation 220 is not present and the planar item 200 remains whole, the eddy currents may be large enough within the planar item 200 to affect the sensing of the field 207 from the EM localizer 94. However, the smaller and/or effectively cancelling augmented fields 210f, 214f due to the augmentation 220 of the planar item 200 may not interfere with sensing the localizer field from the local EM localizer 94. As illustrated in FIG. 3, the induced fields 210f and 214f may generally partially cancel and for specific poses and geometries fully cancel around and/or internal to the tracking device 81. Therefore, the fields 210f, 214f may generally not interfere with sensing of the field 207 from the EM localizer 94. In various embodiments, without being bound by the theory, this may be understood to reducing a size (e.g., an area) of the induced currents and/or making two induced currents instead of one larger one. Near the augmentation, one induced current is moving up and the other is moving down effectively canceling induced currents near the augmentation. Near the augmentation, one induced magnetic field curves clockwise and the other curves counterclockwise effectively cancelling induced fields near the augmentation.

If the tracking device 81 is not being interfered with or sensing an interfering field, the field emitted by the EM localizer 94 may be used to accurately track the tracking device 81. Therefore, regardless of where the planar item 200 is within the field generated by the EM localizer 94 the fields 210f, 214f may not interfere with the sensing of the EM localizer field. This allows the tracking device 81 to be used to determine a pose of a selected device, such as the US probe 33, within a selected or predetermined accuracy, such as within about 0.1 millimeters (mm) to about 10 mm (including tolerances that may be about 0.1 mm) and 0.1 to 10 degrees. In other words, the EM tracking device 81 may only sense the field emitted by the EM localizer 94 for tracking and navigation of the EM tracking device 81 and associate or connected instrument.

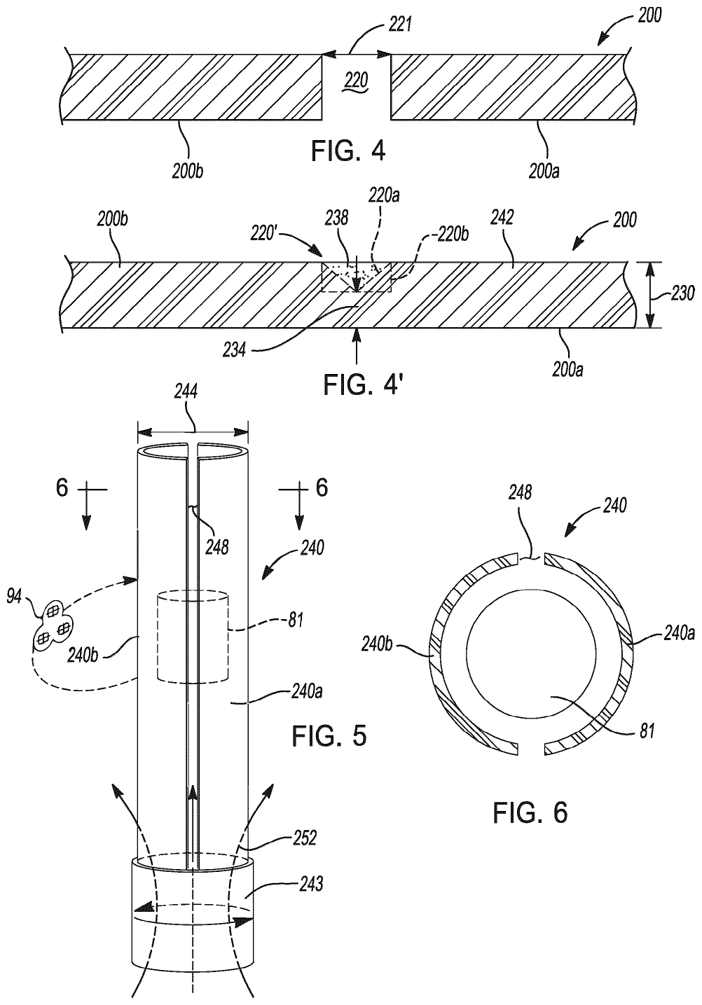

Turning reference to FIG. 4, the planar item 200 having the two portions 200a and 200b is illustrated in greater detail. The augmentation 220 may be a full cut to form a complete separation of the two portions 200a, 200b. The augmentation 220, therefore, generally does not allow for a current to be conducted between the two member portions 200a, 200b. The augmentation 220 may effectively produce a much higher resistance across a gap defined by the augmentation rather than up or down the length of augmentation 220.

Generally, a resistance across arrow 221 the augmentation 220, 220′ (FIGS. 4 and 4′) is greater (e.g., at least twice) than a resistance along arrow 223 the augmentation 220, 220′. Again, without being bound by the theory R=rho*L/A; where R is resistance, rho is the material resistivity, L is the length of the material, and A is the cross-sectional area of the material. For an air gap augmentation 220, A may be considered to be the same where rho_air*L_air>>rho_metal*L_metal. Rho_air is >10\hat{ }8 and exemplary conductive metals may have rho_metal<10\hat{ }-7 so the air gap L_air can be very small. For a thin bridge augmentation 220′ rho may be considered to be the same and A to be W*T (width*thickness [i.e. the rectangular 220b]) with W the same so that L_thin/T_thin>>L_thick/T_thick. With T_thin=T 234 being the width 234 and T_thick=T_230 being the width 230. After rearranging we get L_thin/L_thick>>T_thin/T_thick. Here L_thin may be 1 mm and L_thick may be 10 mm and T_thick may be 1 mm so that T_thin must be <<0.1 mm. One skilled in the art will understand that similar calculations may be used for any appropriate shape, such as a triangular cut 220a.

It is understood that the augmentation 220, even when formed as a complete cut, may have a non-conductive material position therein, such as a non-conductive insulator or other appropriate material that may assist in maintaining a structure integrity of the member 200 or other purposes. Generally, as a complete cut the augmentation is open and non-conductive to prevent conduction across gap of the augmentation 220.

With reference to FIG. 4′, the planar item 200 having the two portions 200a and 200b that may have the augmentation 220′. The augmentation 220′ may include a reduced width of the planar item 200. The augmentation 220′ maybe formed in one or more shapes, such as a triangular cut 220a or a rectangular cut 220b, or combinations thereof. For example, the planar item 200 may include a width, or thickness of 230. The augmentation 220′ may include a thickness 234 that is reduced or less than the thickness 230. The reduced thickness 234 may effectively increase a resistance across the augmentation 220′, particularly to currents induced by the field of the EM localizer 94, such as discussed above. The reduced thickness 234 may substantially eliminate an induced current in the entire planar item 200, even though the augmentation 220′ is not a complete cut through the planar item 200. Nevertheless, the augmentation 200′ may form the member 200 into the two portions 200a, 200b.

The augmentation 200′ having the reduced width or thickness 234 may therefore form a divot or groove 238 below a surface 242 of the planar item 200. The divot 238, however, may be filled with a selected material such as a non-conductive material. Thus, the groove 238 may be filled and formed flush with the surface 242 such that the member 200 is substantially smooth even though the augmentation 220′ has been formed. The surface 242 may be an interior surface or an exterior surface of an instrument, such as the US probe 33.

Accordingly, the member 200 may be formed or have formed therein or thereon a pattern, as discussed above, in the form of an augmentation. The augmentation 220 may, therefore, form the augmented member 200 that may include at least two portions, such as the portions 200a, 200b. It is understood that any appropriate number of augmentations may be provided in the selected pattern such that more than two portions of the planar item 200 may be formed. Further, the augmentation may be in a non-straight pattern. For example, the augmentation may be curved, include selected angles, or be non-continuous along a dimension of the member 200. For example, the augmentation 220 may curve, have an angle, geometric shape, and/or have several passes through the tracking device 81. Further, the planar item 200 may include any appropriate number of augmentations such as including an augmentation 250 and 254. The optional augmentations, according to various embodiments as discussed further herein, may form the planar item or any appropriate member into selected member portions.

Turning to reference to FIG. 5 and FIG. 6, a member or instrument 240 is illustrated. The instrument 240 may have a circular cross section and define a diameter 244. The dimensions of the instrument 240, however, may be any appropriate dimension and may be based upon an instrument, such as the US probe 33. Nevertheless, a current may be induced in the instrument 240 if it includes a portion formed of a conductive material, as discussed above. Therefore, the instrument 240 may also include an augmentation or plurality of augmentations 248. As the EM Localizer 94 generates the field 207, currents induced in the instrument 240 may be minimized or reduced due to the augmentation 248 that may form a first portion 240a and a second portion 240b of the instrument 240. Therefore any currents induced in the portions 240a, 240b may be substantially minimal or reduce. Any currents induced may generate field 252, but it is reduced and/or away from (so as to not interfere with a sensing by) the tracking device 81. For example, a ring of metal 243 may remain intact for other functional (e.g. structural) reasons so that the bottom ring may support an induced current around the bottom ring that makes induced field 252. The tracking device 81 may be positioned in or near the instrument 240. The fields 252 that are generated due to an induced current in that the instrument 240 may be away from the tracking device 81 so as not to interfere with a sensing of the field 207 from the EM Localizer 94.

The tracking device 81 may be positioned at any appropriate position on, in, or near the instrument 240, such as away from the ring 243. As illustrated in FIG. 5, the tracking device 81 may be positioned within the instrument 240. It is understood, however, that the tracking device 81 may also be positioned on a surface of the instrument, or in an appropriate portion of the instrument, and/or positioned a distance away from therefrom. It is understood that interfering fields may interfere with the tracking device 81 if the tracking device 81 is not able to sense the field 207 from the EM localizer, including able to only sense the field from the EM localizer 94. Therefore, the EM tracker 81 may be positioned relative to the augmentation 248 of the instrument 240 to substantially eliminate or reduce any interfering fields due to an induced currents in the instrument 240.

Turning to reference to FIG. 7, the US probe 33 may have the structure or portion (e.g., a sub-housing) 204. The portion 204 may be formed of a conductive material, including any appropriate conductive material such as those discussed above. The US probe 33 includes the ultrasound transducers 125 that may be used to generate an imaging plane 129. The imaging plane 129 may be moved by the user 72 as the user moves the US probe 33 relative to the subject 30. As noted above, however, the US probe 33 may be moved with the robotic arm 20, or any appropriate manner. Regardless, the tracking device 81 may be used to track a pose of the ultrasound probe 33, including the portion 204.

The tracking device 81 may be the EM tracking device that senses the field 207 from the EM Localizer 94. The field 207 may induce eddy currents into the portion 204 such as defined by the circle arrows 260. The eddy currents may generate fields relative to the tracking device 81. The tracking device 81 may be fixed to the portion 204, formed within the portion 204, surrounded by the portion 204, or combinations thereof. However, one or more augmentations, such as an augmentation 270 and/or augmentation 274 may be formed in the portion 204. It is understood that other appropriate number of augmentations may also be formed, such as an augmentation 276 and an augmentation 280. Further, an augmentation 278 may be formed, such as below or adjacent the tracking device 81. Further, the augments may be formed on or near an external surface and/or an internal surface of the housing 204. Also, the tracking device 81 may be associated with the US probe 33, or any appropriate instrument, in any appropriate manner. For example, the tracking device 81 may be positioned on, near, or inside a housing or structure, such as the portion 204, and/or embedded in a portion, such as the portion 204. The tracking device 81 may also be included with the outer housing 205 such as positioned on, near, inside, and/or embedded in the housing 205.

The augmentations, such as the augmentation 270 and 274 may be formed as grooves in the portion 204. As illustrated above, the housing may have a planar surface such that the augmentation includes a separation or groove in the portion 204. The augmentation 270, 274 may be formed by any appropriate tool such as a mill or saw. Further, chemical etching or electrical discharge machining (EDM) may be used to form the augmentation. It is understood that the augmentation 270, 274 may be formed on an external surface of the housing and/or on an internal surface of the portion 204. Accordingly, as illustrated in FIG. 4′, the surface 242 of the planar item 200 may be an external surface or an internal surface. Further, in various embodiments, the augmentation 270, 274 may be formed on both surfaces including an internal and an external surface while still maintaining material in the augmentation area.

Nevertheless, the augmentations, such as the augmentation 270, may confine or limit the size or extent of the induced eddy currents and therefore minimize or reduce any fields generated by the eddy currents. Accordingly, the EM tracking device 81 may substantially only sense the EM field 207 from the EM localizer 94. It is further understood, however, that to the EM tracking device 81 may sense fields from eddy currents formed in the portion 204, but these may be corrected from a determination of a pose of the tracking device 81 and the US probe 33 due to filtering, signal strength, and the like. Various filtering and/or correcting processes may include full or small time constant linearized version of those methods described in U.S. Pat. No. 11,439,317 entitled Position Determination System and Method, incorporated herein by reference.

Accordingly, the augmentations, such as the augmentation 270, may be formed in the portion 204 to allow for tracking of the US probe 33 by use of the EM tracking device 81 by eliminating or at least effectively reducing interfering EM fields (e.g., so as not to interfere with sensing the field 207 of the EM localizer 94). By eliminating or at least effectively reducing an effect of interfering EM fields, the interfering EM fields may be entirely eliminated and/or the signal produced by the EM fields may be easily removed due to a signal strength or location. Thus, the user 72 may move the US probe 33 relative to the subject 30. Thus, the cut or complete opening may generally eliminate a positions for a current to form while a depression of groove may increase an impedance and resistance to reduce a current and related fields.

The user 72 may move the US probe 33 relative to the subject 30. For example, the user 72 may move the US probe 33 relative to the heart 127 of the subject 30. The user 72 may move the probe such that the imaging plane 129 sweeps an area or volume of the subject 30, including the heart 127, collecting discrete image data at each pose at the plane 129. The discrete image data collected at the imaging plane 129 may be displayed with the display 84 as the heart image 127i. In various embodiments, however, a plurality of ultrasound slices or sonograms may be reconstructed into a three-dimensional model of the heart. This reconstructed model or reconstruction may be displayed as the image 127i.

By tracking the US probe 33 with the EM tracking device 81 in the patient space, a determination of the pose of the imaging plane 129 may be determined. Therefore, a plurality of the discrete images collected at each pose of the imaging plane 129, due to the pose of the US probe 33 may be combined in a selected manner to achieve a three-dimensional image. A determination of a pose of the imaging plane 129 relative to the EM tracking device 81 may be determined in an appropriate manner, such as with a calibration system that may include a calibration jig or other appropriate calibration system. The imaging system 33 may be tracked, as discussed above. Various tracking systems may include and/or require calibration of the imaging system. Thus, the pose of the tracking device 22 relative to a plane of the US imaging system may be determined and/or known. Various systems and methods are disclosed in U.S. Pat. Nos. 6,379,302; 6,669,635; 6,968,224; 7,085,400; 7,831,082; 8,320,653; 8,811,662; and 9,138,204 all of which are incorporated herein by reference.

According to various embodiments, the ultrasound probe may emit or transmit ultrasound waves in a selected pattern or plane. The plane may be a shape as is understood by one skilled in the art. The plane is generally able to acquire data in a field of view to generate images, also referred to as sonograms when images are generated based on ultrasound data.

Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

Instructions may be executed by a processor and may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The apparatuses and methods described in this application may be partially or fully implemented by a processor (also referred to as a processor module) that may include a special purpose computer (i.e., created by configuring a processor) and/or a general purpose computer to execute one or more particular functions embodied in computer programs. The computer programs include processor-executable instructions that are stored on at least one non-transitory, tangible computer-readable medium. The computer programs may also include or rely on stored data. The computer programs may include a basic input/output system (BIOS) that interacts with hardware of the special purpose computer, device drivers that interact with particular devices of the special purpose computer, one or more operating systems, user applications, background services and applications, etc.

The computer programs may include: (i) assembly code; (ii) object code generated from source code by a compiler; (iii) source code for execution by an interpreter; (iv) source code for compilation and execution by a just-in-time compiler, (v) descriptive text for parsing, such as HTML (hypertext markup language) or XML (extensible markup language), etc. As examples only, source code may be written in C, C++, C#, Objective-C, Haskell, Go, SQL, Lisp, Java®, ASP, Perl, Javascript®, HTML5, Ada, ASP (active server pages), Perl, Scala, Erlang, Ruby, Flash®, Visual Basic®, Lua, or Python®.

Communications may include wireless communications described in the present disclosure can be conducted in full or partial compliance with IEEE standard 802.11-2012, IEEE standard 802.16-2009, and/or IEEE standard 802.20-2008. In various implementations, IEEE 802.11-2012 may be supplemented by draft IEEE standard 802.11ac, draft IEEE standard 802.11ad, and/or draft IEEE standard 802.11ah.

A processor, processor module, module or ‘controller’ may be used interchangeably herein (unless specifically noted otherwise) and each may be replaced with the term ‘circuit.’ Any of these terms may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

Instructions may be executed by one or more processors or processor modules, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processor module” as used herein may refer to any of the foregoing structure or any other physical structure suitable for implementation of the described techniques. Also, the techniques could be fully implemented in one or more circuits or logic elements. The processor or processors may operate entirely automatically and/or substantially automatically. In automatic operation the processor may execute instructions based on received input and execute instructions in light thereof. Thus, various outputs may be made without further or any manual (e.g., user) input.

The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the invention, and all such modifications are intended to be included within the scope of the invention.

What is claimed is:

1. An instrument system, comprising:
a structure formed of a conductive material, wherein the structure includes a first portion and a second portion separated by an augmentation to at least produce a selected resistance between the first portion and the second portion to at least minimize an induction of a current across the augmentation; and
an electromagnetic (EM) tracking device associated with the structure;
wherein an emitted EM field is operable to induce a field to be emitted from the structure.

2. The system of claim 1, further comprising:
an EM localizer having one or more coils of conductive material;
wherein the emitted EM field is generated by one or more coils of the EM localizer to generate one or more EM fields;
wherein the EM tracking device senses the generated one or more EM field or fields.

3. The system of claim 2, further comprising:
an ultrasound transducer associated with the structure configured to generate an imaging plane for imagining a subject
a navigation processor configured to execute instructions to determine a pose of the imaging plane based at least on the sensed generated one or more EM field or fields.

4. The system of claim 3, wherein the augmentation in the structure is configured to reduce or eliminate a current flow between the first portion and the second portion.

5. The system of claim 4, wherein the augmentation is a separation of the first portion from the second portion.

6. The system of claim 4, wherein the augmentation is a groove formed in the structure.

7. The system of claim 6, wherein the groove is filled with a non-conductive material.

8. The system of claim 1, wherein the selected resistance is across the augmentation is greater than along the augmentation.

9. The system of claim 1, further comprising;
a display device configured to display an image based at least in part on the image data acquired in the image place.

10. A system to track an imaging system and display an image based on acquired image data, comprising:
an electromagnetic (EM) localizer having one or more coils of conductive material, wherein the coil or coils of the EM localizer generates one or more EM fields;
a structure formed of a conductive material, wherein a current is induced in the structure due to the generated one or more EM field or fields;
an EM tracking device configured to sense the generated EM field, wherein the EM tracking device is near the structure;
an ultrasound transducer positioned within the structure and configured to generate an imaging plane for imagine a subject; and
a navigation processor configured to execute instructions to determine a pose of the imaging plane based at least on the sensed generated one or more EM field or fields;
wherein the structure includes a first portion and a second portion separated by an augmentation to at least produce a selected resistance between the first portion and the second portion to at least minimize the induction of the current in the structure.

11. The system of claim 10, wherein the EM tracking device is at least one of fixed to or within the structure.

12. A method of reducing induced magnetic fields of a member, comprising:
providing a conductive member;
providing an augmentation in the conductive member to increase a resistance between a first portion of the conductive member and a second portion of the conductive member; and
configuring the first portion of the conductive member and the second portion of the conductive to at least reduce an induction of magnetic fields of the conductive member.

13. The method of claim 12, further comprising:
providing an EM tracking device configured to sense a generated one or more EM field or fields;
providing the member as a structure;
providing the EM tracking device near the structure; and
providing an ultrasound transducer within the structure.

14. The method of claim 13, further comprising:
operating the ultrasound transducer to generate an imaging plane to imagine a subject.

15. The method of claim 13, further comprising:
operating an EM localizer to generate the one or more EM field or fields; and
operating a navigation processor to execute instructions to determine a pose of the imaging plane based at least on a sensing of the generated one or more EM field or fields by the EM tracking device.

16. The method of claim 15, further comprising:
determining a pose of a plurality of image slices based at least on the determined pose of the imaging plane over time.

17. The method of claim 16, further comprising:
moving the imaging plane over the time.

18. The method of claim 13, further comprising:
at least one of fixing the EM tracking device to the structure or positing the EM tracking device within the structure.
